(19) United States
(12) Patent Application Publication
(10) Pub. No.: US 2026/0123865 A1
(43) Pub. Date: May 7, 2026

(54) A METHOD FOR SAMPLING A BODILY FLUID FROM A TEST SUBJECT, AND A SAMPLING SYSTEM

(71) Applicant: Ascilion AB, Kista (SE)

(72) Inventors: Markus Renlund, Åkersberga (SE); Pelle Rangsten, Storvreta (SE)

(73) Assignee: Ascilion AB, Kista (SE)

(21) Appl. No.: 19/119,937

(22) PCT Filed: Oct. 13, 2023
(86) PCT No.: PCT/SE2023/051021

§ 371 Date: Apr. 10, 2025

(30) Foreign Application Priority Data
Oct. 13, 2022 (SE) 2251200-8

(51) Int. Cl. A61B 5/15

(52) U.S. Cl. CPC A61B 5/150984; A61B 5/150099

(57) ABSTRACT

The present invention relates to a method for sampling a bodily fluid from the skin of a test subject by means of a sampling system The method comprises the steps of a) placing the rim of the microneedle assembly in contact with the skin of the test subject, thereby sealing the interior of the housing from the surrounding atmosphere; b) applying a negative penetration pressure by means of the pump to the fluid port and thereby to the interior of the housing, wherein the negative penetration pressure is maintained during a penetration time, whereby the plurality of microneedles penetrates the skin of the test subject; c) lift-off, wherein the plurality of microneedles are removed from the skin during a lift-off time wherein the interior of the housing is at least at the atmospheric pressure; and d) applying negative extraction pressure with the pump during an extraction time whereby the bodily fluid released from the skin flows towards the fluid port and is collected in a sample volume The present invention also relates to a sampling system.

### TECHNICAL FIELD

The present disclosure generally relates to the field of sampling a fluid from test subject and more specifically to the field of sampling a bodily fluid with a microneedle array.

### BACKGROUND

In modern analysis of test subjects such as for example human objects or animals there is a large interest in analyzing different bodily fluids and mixtures thereof. Traditionally, for a diabetes patient that wants to test the blood sugar level a lancet were used to penetrate the skin and extract a small amount of blood through the opening in the skin and then subject the test strip to the blood coming out of the skin were the lancet entered the skin. In recent years, a large interest has emerged in sampling other bodily fluids that are possible to extract from the skin of the test subject without causing damage to the skin and pain to the test subject.

A common problem associated with sampling of bodily fluids is that the volume of bodily fluid is rather small and troublesome to extract in an efficient way.

Some attempts have been performed to efficiently extract a bodily fluid from a test subject by introducing a local edema, which is used for sampling of the bodily fluid. This is for example disclosed in US20200315502. This method is both painful and provides a long lasting discoloring of the skin. It is therefore of great interest if an alternative method could be identified.

Therefore, it is of great interest to provide a method for sampling of a bodily fluid that is able to efficiently extract the bodily fluid without introducing a local edema and still being effective.

### SUMMARY

An object of the present disclosure is to provide a sampling method which seeks to mitigate, alleviate, or eliminate one or more of the above-identified deficiencies in the art and disadvantages singly or in any combination and to provide an improved sampling method.

A further object of the present disclosure is to provide an improved sampling system.

This object is obtained by a method for sampling a bodily fluid from the skin of a test subject by means of a sampling system, wherein the sampling system comprises: a fluid pump; a microneedle assembly; a conduit connecting the fluid pump to the microneedle assembly; the microneedle assembly comprises: a housing comprising a fluid port connected to the fluid pump via the conduit; a microneedle array in fluid communication with the fluid port, wherein the microneedle array is formed on a substrate and comprises a plurality of microneedles extending from a first side of the substrate, each microneedle having a proximal end connected to the first side of the substrate and a distal end, wherein the distal end has a bevel, and wherein each needle has a hole extending from its proximal end to its distal end, and wherein each hole extends into a corresponding hole of the substrate, wherein the plurality of the microneedles provides a fluid path from the distal end to the fluid port; a rim extending around the microneedle array wherein the rim is aimed to define a sealing between the surrounding atmosphere and an interior of the housing when the rim is in contact with the skin of the test subject; wherein the method comprises the steps of a) placing the rim of the microneedle assembly in contact with the skin of the test subject, thereby sealing the interior of the housing from the surrounding atmosphere; b) applying a negative penetration pressure by means of the pump to the fluid port and thereby to the interior of the housing, wherein the negative penetration pressure is maintained during a penetration time, whereby the plurality of microneedles penetrates the skin of the test subject; c) lift-off, wherein the plurality of microneedles are removed from the skin during a lift-off time, wherein the interior of the housing is at least at the atmospheric pressure; and d) applying a negative extraction pressure with the pump during an extraction time, whereby the bodily fluid released from the skin flows towards the fluid port and is collected in a sample volume.

The object is also obtained by a sampling system for sampling bodily fluid from the skin of a test subject wherein the sampling system comprises: a fluid pump; a microneedle assembly; a conduit connecting the fluid pump to the microneedle assembly; the microneedle assembly comprises a housing comprising a fluid port connected to the fluid pump via the conduit; a microneedle array in fluid communication with the fluid port, wherein the microneedle array is formed on a substrate and comprises a plurality of microneedles extending from a first side of the substrate, each microneedle having a proximal end connected to the first side of the substrate and a distal end, wherein the distal end has a bevel, and wherein each needle has a hole extending from its proximal end to its distal end, and wherein each hole extends into a corresponding hole of the substrate, wherein the plurality of the microneedles provides a fluid path from the distal end to the fluid port; a rim extending around the microneedle array, wherein the rim is aimed to define a sealing between the surrounding atmosphere and an interior of the housing when the rim is in contact with the skin of the test subject; a control unit configured to control the fluid pump and comprising a pressure sensor arranged to measure the pressure in the flow path through the conduit, wherein the control unit is configured to perform a method as set out in embodiments disclosed herein.

### BRIEF DESCRIPTION OF THE DRAWINGS

The foregoing will be apparent from the following more particular description of the example embodiments, as illustrated in the accompanying drawings in which like reference characters refer to the same parts throughout the different views. The drawings are not necessarily to scale, emphasis instead being placed upon illustrating the example embodiments.

FIG. 1 is a schematic block diagram of a sampling system according to one embodiment of the present invention.

FIG. 2 is flow diagram illustrating an embodiment of a method according to one embodiment of the present invention.

FIG. 3 is a pressure vs. time diagram illustrating an embodiment of the present invention.

FIG. 4 is a schematic block diagram of a control unit according to one embodiment of the present invention.

### DETAILED DESCRIPTION

Aspects of the present disclosure will be described more fully hereinafter with reference to the accompanying drawings. The apparatus and method disclosed herein can, however, be realized in many different forms and should not be construed as being limited to the aspects set forth herein. Like numbers in the drawings refer to like elements throughout.

The terminology used herein is for the purpose of describing particular aspects of the disclosure only, and is not intended to limit the invention. As used herein, the singular forms “a”, “an” and “the” are intended to include the plural forms as well, unless the context clearly indicates otherwise.

In this disclosure the term ‘bodily fluid’ should be interpreted as a fluid extracted from the skin of a test object. This fluid may for example be interstitial fluid or a mixture of interstitial fluid and other fluids present in the skin such as plasma or intracellular fluid.

In this disclosure the term ‘test subject’ should be interpreted to encompass both humans and animals, as well as ex vivo tissue for experimental purposes.

Some of the example embodiments presented herein are directed towards a method for sampling a bodily fluid. As part of the development of the example embodiments presented herein, a problem will first be identified and discussed.

In some earlier attempts to extract a bodily fluid with a microneedle array (MNA) an under pressure were applied to fluid channels of the MNA in order to provide a suction effect. The present inventors tried many different under pressures in order to improve the flow of bodily fluid from a test subject. However, none of these experiments proved to be a success and only limited sample volumes were extracted.

The present inventors realized that these problems may be minimized or even eliminated by introducing a pressure sequence to the MNA starting with a penetration step wherein a negative penetration pressure is applied to the MNA. This step is followed by a lift-off step where the MNA is lifted from the skin either by a positive pressure, or by ventilating the under pressure and mechanically lift the MNA from the skin. This lift-off step is then followed by an extraction step where an under pressure is applied. This sequence provided an unexpected and surprisingly increased flow of bodily fluid from the skin.

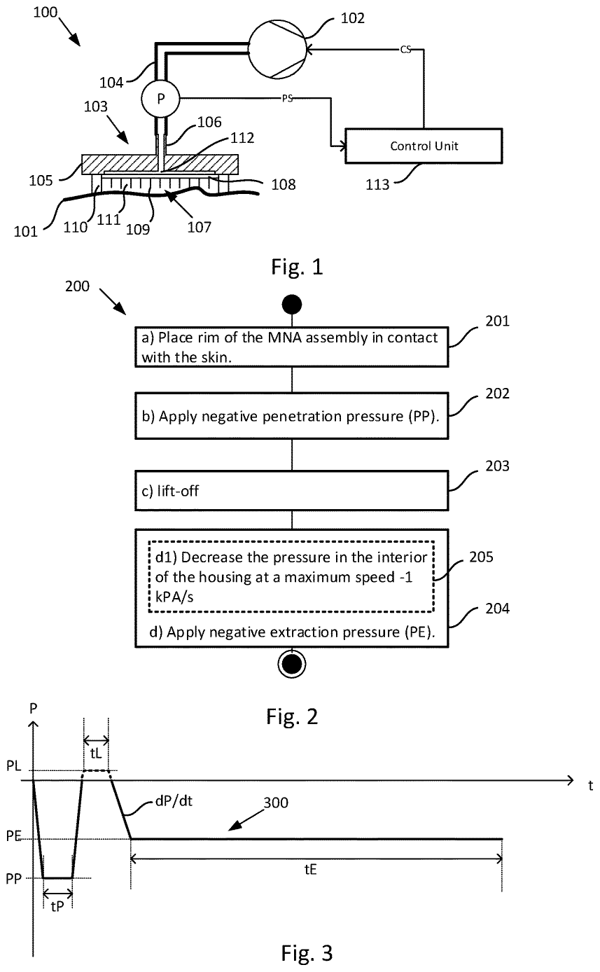

Now with reference made to FIG. 1 and FIG. 2 and FIG. 3 a method for sampling a bodily fluid from the skin of a test subject by means of a sampling system will be disclosed. The sampling system 100 comprises: a fluid pump 102; a microneedle assembly 103; a conduit 104 connecting the fluid pump 102 to the microneedle assembly 103.

The microneedle assembly 103 comprises: a housing 105 comprising a fluid port 106 connected to the fluid pump 102 via the conduit 104; a microneedle array 107 in fluid communication with the fluid port 106.

The microneedle array 107 is formed on a substrate 108 and comprises a plurality of microneedles extending from a first side of the substrate, each microneedle 109 having a proximal end connected to the first side of the substrate 108 and a distal end. The distal end has a bevel, and wherein each needle has a hole extending from its proximal end to its distal end, and wherein each hole extends into a corresponding hole of the substrate. The plurality of the microneedles provides a fluid path from the distal end to the fluid port.

The microneedle assembly 103 further comprises a rim 110 extending around the microneedle array 107 wherein the rim is aimed to define a sealing between the surrounding atmosphere and an interior 111 of the housing when the rim is in contact with the skin of the test subject.

The method, generally designated 200 in FIG. 2, comprises the steps of:
Step a) Placing 201 the rim of the microneedle assembly in contact with the skin of the test subject, thereby sealing the interior 111 of the housing 105 from the surrounding atmosphere.
Step b) Applying 202 a negative penetration pressure, PP, by means of the pump to the fluid port and thereby to the interior of the housing, wherein the negative penetration pressure, PP, is maintained during a penetration time, tP, whereby the plurality of microneedles penetrates the skin of the test subject.
Step c) Lift-off 203, wherein the plurality of microneedles are removed from the skin during a lift-off time tL, wherein the interior of the housing is at least at the atmospheric pressure; and
Step d) applying 204 a negative extraction pressure PE with the pump during an extraction time tE, whereby the bodily fluid released from the skin flows towards the fluid port and is collected in a sample volume 112.

The negative penetration pressure PP is in the interval from <30 kPa to −45 kPa.

The penetration time tP is in the interval from 10 s to 60 s.

The negative extraction pressure PE is in the interval from −10 kPa to −30 kPA.

The negative extraction pressure PE is in the interval from −20 kPa to −30 kPa. In one embodiment the negative extration pressure is −25 kPa.

Optionally, step d) 204 further comprises a step d1) 205 of decreasing the pressure in the volume during lift-off to the negative extraction pressure at a pressure difference speed smaller than 1 kPa/s.

Now once again with reference made to FIG. 1 the sampling system 100 further comprises a control unit 113 configured to control the fluid pump and comprising a pressure sensor arranged to measure the pressure PS in the flow path through the conduit 104, wherein the control unit 113 is configured to perform a method 200 as set out in embodiments disclosed herein.

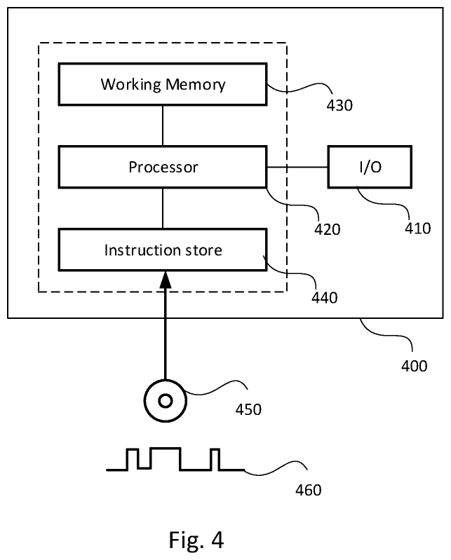

FIG. 4 shows an exemplary implementation of the control unit 113, in programmable signal processing hardware. The signal processing apparatus 400 shown in FIG. 4 comprises an input/output (I/O) section 410 for receiving the measured pressure, and transmitting control signals, CS, to the pump 102. The signal processing apparatus 400 further comprises a processor 420, a working memory 430 and an instruction store 440 storing computer-readable instructions which, when executed by the processor 420, cause the processor 420 to perform the processing operations herein described to control the sampling system 100. The instruction store 440 may comprise a ROM which is pre-loaded with the computer-readable instructions. Alternatively, the instruction store 440 may comprise a RAM or similar type of memory, and the computer readable instructions can be input thereto from a computer program product, such as a computer-readable storage medium 450 such as a CD-ROM, etc. or a computer-readable signal 460 carrying the computer-readable instructions.

In the present embodiment, the combination 470 of the hardware components shown in FIG. 4, comprising the processor 420, the working memory 430 and the instruction store 440, is configured to implement the functionality of the aforementioned control unit 113.

The disclosure relates to a method for sampling a bodily fluid from the skin of a test subject by means of a sampling system, wherein the sampling system comprises: a fluid pump; a microneedle assembly; a conduit connecting the fluid pump to the microneedle assembly; the microneedle assembly comprises: a housing comprising a fluid port connected to the fluid pump via the conduit; a microneedle array in fluid communication with the fluid port, wherein the microneedle array is formed on a substrate and comprises a plurality of microneedles extending from a first side of the substrate, each microneedle having a proximal end connected to the first side of the substrate and a distal end, wherein the distal end has a bevel, and wherein each needle has a hole extending from its proximal end to its distal end, and wherein each hole extends into a corresponding hole of the substrate, wherein the plurality of the microneedles provides a fluid path from the distal end to the fluid port; a rim extending around the microneedle array wherein the rim is aimed to define a sealing between the surrounding atmosphere and an interior of the housing when the rim is in contact with the skin of the test subject; wherein the method comprises the steps of a) placing the rim of the microneedle assembly in contact with the skin of the test subject, thereby sealing the interior of the housing from the surrounding atmosphere; b) applying a negative penetration pressure by means of the pump to the fluid port and thereby to the interior of the housing, wherein the negative penetration pressure is maintained during a penetration time, whereby the plurality of microneedles penetrates the skin of the test subject; c) lift-off, wherein the plurality of microneedles are removed from the skin during a lift-off time, wherein the interior of the housing is at least at the atmospheric pressure; and d) applying a negative extraction pressure with the pump during an extraction time, whereby the bodily fluid released from the skin flows towards the fluid port and is collected in a sample volume.

According to some embodiments, the negative penetration pressure is in the interval from −30 kPa to −45 kPa.

According to some embodiments, the penetration time is in the interval from 10 s to 60 s.

According to some embodiments, the negative extraction pressure is in the interval from −10 kPa to −30 kPA.

According to some embodiments, the negative extraction pressure is in the interval from −20 kPa to −30 kPa.

According to some embodiments, the step d) further comprises a step d1) of decreasing the pressure in the volume during lift-off to the negative extraction pressure at a pressure difference speed smaller than 1 kPa/s.

The disclosure also relates to a sampling system for sampling bodily fluid from the skin of a test subject. The sampling system comprises: a fluid pump; a microneedle assembly; a conduit connecting the fluid pump to the microneedle assembly; the microneedle assembly comprises a housing comprising a fluid port connected to the fluid pump via the conduit; a microneedle array in fluid communication with the fluid port, wherein the microneedle array is formed on a substrate and comprises a plurality of microneedles extending from a first side of the substrate, each microneedle having a proximal end connected to the first side of the substrate and a distal end, wherein the distal end has a bevel, and wherein each needle has a hole extending from its proximal end to its distal end, and wherein each hole extends into a corresponding hole of the substrate, wherein the plurality of the microneedles provides a fluid path from the distal end to the fluid port; a rim extending around the microneedle array, wherein the rim is aimed to define a sealing between the surrounding atmosphere and an interior of the housing when the rim is in contact with the skin of the test subject; a control unit configured to control the fluid pump and comprising a pressure sensor arranged to measure the pressure in the flow path through the conduit, wherein the control unit is configured to perform a method as set out in embodiments disclosed herein.

In the drawings and specification, there have been disclosed exemplary embodiments. However, many variations and modifications can be made to these embodiments. Accordingly, although specific terms are employed, they are used in a generic and descriptive sense only and not for purposes of limitation, the scope of the embodiments being defined by the following claims.

The description of the example embodiments provided herein have been presented for purposes of illustration. The description is not intended to be exhaustive or to limit example embodiments to the precise form disclosed, and modifications and variations are possible in light of the above teachings or may be acquired from practice of various alternatives to the provided embodiments. The examples discussed herein were chosen and described in order to explain the principles and the nature of various example embodiments and its practical application to enable one skilled in the art to utilize the example embodiments in various manners and with various modifications as are suited to the particular use contemplated. The features of the embodiments described herein may be combined in all possible combinations of methods, apparatus, modules, systems, and computer program products.

It should be appreciated that the example embodiments presented herein may be practiced in any combination with each other. It should be noted that the word “comprising” does not necessarily exclude the presence of other elements or steps than those listed and the words “a” or “an” preceding an element do not exclude the presence of a plurality of such elements. It should further be noted that any reference signs do not limit the scope of the claims, that the example embodiments may be implemented at least in part by means of both hardware and software, and that several “means”, “units” or “devices” may be represented by the same item of hardware.

1. A method for sampling a bodily fluid from the skin of a test subject by means of a sampling system, wherein the sampling system comprises:
a fluid pump;
a microneedle assembly;
a conduit connecting the fluid pump to the microneedle assembly;
the microneedle assembly comprises:
a housing comprising a fluid port connected to the fluid pump via the conduit;
a microneedle array in fluid communication with the fluid port, wherein the microneedle array is formed on a substrate and comprises a plurality of microneedles extending from a first side of the substrate, each microneedle having a proximal end connected to the first side of the substrate and a distal end, wherein the distal end has a bevel, and wherein each needle has a hole extending from its proximal end to its distal end, and wherein each hole extends into a corresponding hole of the substrate, wherein the plurality of the microneedles provides a fluid path from the distal end to the fluid port;
a rim extending around the microneedle array, wherein the rim is aimed to define a sealing between the surrounding atmosphere and an interior of the housing when the rim is in contact with the skin of the test subject;
wherein the method comprises the steps of:
a) placing the rim of the microneedle assembly in contact with the skin of the test subject, thereby sealing the interior of the housing from the surrounding atmosphere;
b) applying a negative penetration pressure by means of the pump to the fluid port and thereby to the interior of the housing, wherein the negative penetration pressure is maintained during a penetration time, whereby the plurality of microneedles penetrates the skin of the test subject;
c) lift-off, wherein the plurality of microneedles are removed from the skin during a lift-off time wherein the interior of the housing is at least at the atmospheric pressure; and
d) applying a negative extraction pressure with the pump during an extraction time whereby the bodily fluid released from the skin flows towards the fluid port and is collected in a sample volume.

2. A method according to claim 1, wherein the negative penetration pressure is in the interval from −30 kPa to −45 kPa.

3. A method according to claim 1, wherein the penetration time is in the interval from 10 s to 60 s.

4. A method according to claim 1, wherein the negative extraction pressure is in the interval from −10 kPa to −30 kPA.

5. A method according to claim 1, wherein the negative extraction pressure is in the interval from −20 kPa to −30 kPa.

6. A method according to claim 1, wherein step d) further comprises a step d1 of decreasing the pressure in the volume during lift-off to the negative extraction pressure at a pressure difference speed smaller than 1 kPa/s.

7. A sampling system for sampling bodily fluid from the skin of a test subject, wherein the sampling system comprises:
a fluid pump;
a microneedle assembly
a conduit connecting the fluid pump to the microneedle assembly the microneedle assembly comprises:
a housing comprising a fluid port connected to the fluid pump via the conduit;
a microneedle array in fluid communication with the fluid port wherein the microneedle array is formed on a substrate and comprises a plurality of microneedles extending from a first side of the substrate, each microneedle having a proximal end connected to the first side of the substrate and a distal end, wherein the distal end has a bevel, and wherein each needle has a hole extending from its proximal end to its distal end, and wherein each hole extends into a corresponding hole of the substrate, wherein the plurality of the microneedles provides a fluid path from the distal end to the fluid port;
a rim extending around the microneedle array wherein the rim is aimed to define a sealing between the surrounding atmosphere and an interior of the housing when the rim is in contact with the skin of the test subject;
a control unit configured to control the fluid pump and comprising a pressure sensor arranged to measure the pressure in the flow path through the conduit wherein the control unit
upon placing the rim of the microneedle assembly in contact with the skin of the test subject, thereby sealing the interior of the housing from the surrounding atmosphere, is configured to:
a) apply a negative penetration pressure by means of the pump to the fluid port and thereby to the interior of the housing, wherein the negative penetration pressure is maintained during a penetration time, whereby the plurality of microneedles penetrates the skin of the test subject;
b) increase the pressure in the volume, lift-off, wherein the plurality of microneedles are removed from the skin during a lift-off time wherein the interior of the housing is at least at the atmospheric pressure; and
d) apply a negative extraction pressure with the pump during an extraction time, whereby the bodily fluid released from the skin flows towards the fluid port and is collected in a sample volume

8. A sampling system according to claim 7, wherein the negative penetration pressure is in the interval from −30 kPa to −45 kPa.

9. A sampling system according to claim 7, wherein the penetration time is in the interval from 10 s to 60 s.

10. A sampling system according to claim 7, wherein the negative extraction pressure is in the interval from −10 kPa to −30 kPA.

11. A sampling system according to claim 7, wherein the negative extraction pressure is in the interval from −20 kPa to −30 kPa.

12. A sampling system according to claim 7, wherein step d) further comprises a step d1 of decreasing the pressure in the volume during lift-off to the negative extraction pressure at a pressure difference speed smaller than 1 kPa/s.
